(19) United States
(12) Patent Application Publication
(10) Pub. No.: US 2017/0241497 A1
(43) Pub. Date: Aug. 24, 2017

(54) Planar Torsion Spring for Knee Prostheses and Exoskeletons

(71) Applicant: Massachusetts Institute of Technology, Cambridge, MA (US)

(72) Inventors: Luke Mooney, Westford, MA (US); Kenneth Alan Pasch, Dover, MA (US); Thuan D. Doan, Gardena, CA (US)

(21) Appl. No.: 15/402,186

(22) Filed: Jan. 9, 2017

Related U.S. Application Data
(60) Provisional application No. 62/276,781, filed on Jan. 8, 2016.

(51) Int. Cl. F16F 1/02; A61H 1/02; A61F 2/64

(52) U.S. Cl. CPC F16F 1/027; F16F 1/021; A61F 2/64; A61H 1/024; F16F 2228/001; F16F 2234/06; F16F 2226/04; A61F 2002/5072

(57) ABSTRACT

A planar torsion spring has outer and inner hubs connected by a set of beams that are capable of bending to provide torsional compliance when the outer hub is rotated with respect to the inner hub. Each beam is fixed to the outer hub at one end and is attached to the inner hub at its other end by a pin and slot. Slots may be curved. The spring is capable of deflecting to ±

$$\frac{π}{6}$$

radians and providing 100 N·m of torque. Bearings may be located at the interface between each pin and slot. Beams may have variable width. In a method of fabrication, the design dimensions, material, and slot geometry of the planar torsion spring can be parameterized to design springs that meet specific requirements for different applications. In addition to quantifying performance, the models provide the foundation for further weight, efficiency, and performance optimization.

### RELATED APPLICATIONS

This application claims the benefit of U.S. Provisional Application Ser. No. 62/276,781, filed Jan. 8, 2016, the entire disclosure of which is herein incorporated by reference.

### FIELD OF THE TECHNOLOGY

The present invention relates to robotic actuators and, in particular, to a planar torsion spring for use in prosthesis and exoskeletons.

### BACKGROUND

As the fields of rehabilitation robotics, legged robots, prostheses, and exoskeletons continue to grow, series elastic actuators (SEAs) are increasingly utilized. Because applications where the compliance provided by an SEA is desired are so diverse, much research in the past decade has been dedicated to developing custom SEAs to meet the specific requirements of different applications. However, due to the mechanical complexity of a passive, elastic element, existing SEAs are typically heavy, bulky, and not well-suited for applications where there exist strict weight and form-factor constraints, such as exoskeletons and prostheses.

In general, a series elastic actuator (SEA) consists of a stiff actuator with a spring in series between the actuator and the load. While a stiff actuator operating independently is capable of moving to and maintaining desired positions or following predefined trajectories, an SEA will allow deviation from an equilibrium position [Ronald Van Ham, Thomas G. Sugar, Bram Vanderborght, Kevin W. Hollander, and Dirk Lefeber. Complaint actuator designs. Institute of Electrical and Electronics Engineers Journal, 2009]. Stiff actuators are well-suited for position-controlled applications where accurate point and trajectory tracking is required, but are less-suited for applications where spring-like behavior similar to those found in biological systems are desired [Gill A. Pratt and Matthew M. Williamson. Series elastic actuators. Institute of Electrical and Electronics Engineers Journal, 1995].

Compared to stiff actuators, the compliance afforded by SEAs allows exoskeleton and rehabilitation robotic systems to absorb large positional errors that occur due to human-system interfaces, preventing damage to the system and injury to the user [S. Arumugom, S. Muthuraman, and V. Ponselvan. Modeling and application of series elastic actuators for force control multi-legged robots. Journal of Computing, 1, December 2009]. The elastic element allows energy to be stored and released mechanically, which is more efficient than using electric actuators as generators [Gill A. Pratt and Matthew M. Williamson. Series elastic actuators. Institute of Electrical and Electronics Engineers Journal, 1995]. Furthermore, in legged robotics and rehabilitation applications, SEAs reduce shock loading on the transmission that may occur during operation.

The smoothness of force transmission of the actuator becomes much less significant since the series elasticity acts as a transducer between the actuator output position and load force. As a result, the actuator's required force fidelity is decreased while force control stability is improved [Jerry Pratt, Ben Krupp, and Chris Morse. Series elastic actuators for high fidelity force control. Industrial Robot: An International Journal, 29, 2002]. In force control applications, the deflection of the elastic element can be measured and used as a feedback mechanism in force controllers. [Ronald Van Ham, Thomas G. Sugar, Bram Vanderborght, Kevin W. Hollander, and Dirk Lefeber. Complaint actuator designs. Institute of Electrical and Electronics Engineers Journal, 2009].

The existing elastic elements for SEAs can be categorized into three main groups: planar springs, mechanisms that utilize an arrangement of compression springs, and more complex stiffness-controlled systems. While there is a relatively large diversity of planar torsion spring designs, they are all typically monolithic springs that store energy in beam bending as the outer hub rotates with respect to the inner hub. Planar torsion springs can be configured in parallel or series to meet the differing requirements for specific applications. Compression springs mechanisms provide an alternative approach to providing rotary compliance by employing a configuration of linear springs. Stiffness-controlled systems include the large number of custom controllable stiffness actuators that have been designed for various robotic applications. These include equilibrium-controlled stiffness, antagonistic-controlled stiffness, and structure-controlled stiffness actuators. A specific variable stiffness actuator design can be one in which three pulleys and two servo motors are used to control equilibrium position and actuator stiffness [Ronald Van Ham, Thomas G. Sugar, Bram Vanderborght, Kevin W. Hollander, and Dirk Lefeber. Complaint actuator designs. Institute of Electrical and Electronics Engineers Journal, 2009].

While a single elastic element may not satisfy both the torque and deflection requirements, in evaluating the spring design, the existing NASA planar torsion spring [U.S. Pat. No. 8,176,806; Chris A. Ihrke, Adam H. Parsons, Joshua S. Mehling, and Bryan K. Griffith; Planar torsion spring; May 15, 2012] is used as a baseline from which performance metrics are compared. This torsion spring has a generally planar, disc shape and was developed by NASA for use with a robotic arm. It features concentric inner and outer hubs that are connected by splines, having an outer mounting hub that is concentric to the inner mounting hub from which two splines extend radially. The splines vary in width with the length, having a decreased average width towards the middle of the segment. The inner hub is actively rotated by an actuator or drive components, rotating it to move relative to the outer segment, which is attached to the robotic arm. Aspects of this design, such as the spring width, spline widths, spline shape, and material can be changed to obtain the stiffness desired for different applications.

Each of the discussed torsion spring designs have their advantages and disadvantages with regards to size, versatility, adapatability, dynamics, and torque response.

### SUMMARY

The present invention is a novel torsion spring for use in a knee-joint exoskeleton or prostheses. A torsion spring according to the invention is capable of higher angular deflections than previous planar torsion springs, able to withstand high torques, and has a much more compact form factor than previous solutions. Through a fully parametrized model, the effects of material, beam width, beam thickness, and slot design on efficiency, torque response and deflection are better understood. The model also permits further optimization of the spring size, weight, max stresses, and efficiency. This permits various aspects of the spring, such as non-linear deflection characteristic, to be customized to meet requirements for specific applications.

A planar torsion spring according to the invention provides an alternative to the elastic elements currently used in series elastic actuators. In particular, a torsion spring according to the invention provides an alternative torsionally elastic solution that has the ability to undergo comparatively higher angular deflections, while still maintaining a compact form factor, which is desirable in a variety of applications including exoskeletons, prostheses, and rehabilitation robotics. The spring according to the invention opens up an entire design space with potential optimization and performance trade-offs that existing fixed, fixed beam torsion springs lack.

In one aspect, a planar torsion spring according to the invention includes an outer hub, an inner hub, and a plurality of beams connecting the outer hub to the inner hub, wherein the beams are capable of undergoing sufficient bending to provide torsional compliance when the outer hub is rotated with respect to the inner hub and each beam is fixed to the outer hub at one end of the beam and is attached to the inner hub at the other end of the beam by a respective pin and a slot. In some embodiments, the slots may be curved. In a preferred embodiment, there are more than two beams. The planar torsion spring is capable of deflecting greater than ±

$$\frac{π}{36}$$

radians, and is preferably capable of deflecting to at least ±

$$\frac{π}{6}$$

radians. The spring is preferably capable of providing at least 100 N·m of torque. The spring may be made of maraging steel. The spring may include a bearing located at the interface between each pin and slot. At least some of the beams may have a variable width along their length or may have a different width than other beams.

In another aspect of the invention, a method for fabricating an application-specific planar torsion spring according to a set of application-based constraints includes the steps of: based on the application-based constraints, parameterizing at least some of beam width, beam length, beam thickness, beam material, and slot geometry of the planar torsion spring to obtain a parameterized model that characterizes the effects of the parameters on efficiency, torque response, and deflection; based on the parameterized model, establishing an initial design; optimizing the initial design for at least some of weight, size, maximum stresses, stiffness, efficiency, and performance in order to obtain an optimized torsion spring design; and fabricating the planar torsion spring according to the optimized torsion spring design.

In some embodiments, the spring thickness may be adjusted to obtain the desired stiffness and torque. The amount of material in the spring may be minimized while maximizing energy storage. The amount of stiffness in loading the spring may be minimized while maximizing deflection. The step of parameterizing may include mathematical modeling of beam bending to determine beam boundary conditions that maximize deflection before yielding. The beam boundary conditions may include a fixed, fixed-roller beam, a fixed, pin-roller beam, and a fixed, free beam. Analysis may be performed on the amount of stress, bending energy, and tensile energy in each beam. The amount of maximum beam stress when the beams are undergoing both bending and loading may be calculated by superposition of the axial and bending stresses in each beam. The stiffness of each beam may be calculated by taking the numerical derivative of the energy stored in each beam. The step of optimizing may include calculating the forces acting on at least one of the pins and the slots in order to determine the torque response of the spring.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 depicts an example of a preferred embodiment of a planar torsion spring according to the invention.

FIG. 2 depicts several different prototype embodiments of planar torsion springs according to the invention.

FIG. 3 depicts a prototype that includes two torsional springs in series.

FIG. 4 is a graph of the results obtained from testing the prototype of FIG. 3 with a torque sensor and rotation sensor in order to measure stiffness and hysteresis.

FIG. 5 is a graph showing the modulus of resistance for various materials which were compared during the design process.

FIGS. 6A-C depict three alternate beam bending boundary conditions, wherein FIG. 6A is a Fixed, Fixed-Roller Beam, FIG. 6B is Fixed, Pin-Roller Beam, and FIG. 6C is a Fixed, Free Beam.

FIG. 7 is a graph showing the deflection profile of a fixed, fixed-roller beam according to FIG. 6A, undergoing 0.01 meters of deflection.

FIG. 8 is a graph showing the deflection profile of a fixed, pinned-roller beam according to FIG. 6B, undergoing 0.01 meters of deflection.

FIG. 9 is a graph showing the deflection profile of a fixed, free cantilever beam according to FIG. 6C, undergoing 0.01 meters of deflection.

FIG. 10 depicts how, using superposition of axial and bending stresses in a beam undergoing both bending and tensile loading, the resulting maximum stress in each beam condition can be calculated.

FIG. 11 is a graph depicting the max stress in each of the beams of FIGS. 6A-C, calculated as a function of deflection.

FIG. 12 is a graph depicting the beam force in each of the beams of FIGS. 6A-C, calculated as a function of deflection.

FIG. 13 is a graph depicting the effect of axial loading on each of the beams of FIGS. 6A-C, with the stiffness of each of the beams being calculated as a function of deflection.

FIG. 14 is a graph demonstrating that the stiffness of the fixed, free cantilever beam is constant, being independent of deflection.

FIG. 15 is a basic schematic of the beam and inner hub radius for a pinned, straight-slot beam design.

FIG. 16 depicts how the trajectory of the beam tip as it bends is calculated as a function of beam tip angle, deflection, and beam elongation due to bending.

FIG. 17 is a graph showing the trajectory of the beam tip undergoing 0.01 meters of deflection.

FIG. 18 depicts how, as the inner hub undergoes angular deflection, the resulting beam deflection changes the radius vector on which the torque is acting.

FIG. 19 depicts the forces that act on the pin at the tip of the beam.

FIG. 20 depicts the forces due to each body acting on the pin, decomposed into their respective parts.

FIG. 21 depicts the forces acting on the slot, decomposed into their respective parts.

FIG. 22 is a graph showing the torque response for turning the spring and then returning it to equilibrium for a 10-beam spring.

FIG. 23 is a graph showing the efficiency of the spring as a function of angular rotation on the inner hub for various coefficients of friction.

FIG. 24 is a graph showing the torque response of a 10-beam spring for various coefficients of friction.

FIG. 25 shows that the curvature of the slot is such that, at any given θₜᵤᵣₙ, the angle between the slot at that point and the radius vector, θₛₗₒₜ, is constant.

FIG. 26 depicts a curved slot design resulting from the results depicted in FIG. 25.

FIG. 27 depicts how forces act on a slot that is angled with respect to the radius vector.

FIG. 28 is an efficiency contour plot showing the effect of θₜᵤᵣₙ and θₛₗₒₜ on the efficiency of the spring.

### DETAILED DESCRIPTION

A novel torsion spring design for use in knee prostheses and exoskeletons is a planar spring design that features an outer hub and an inner hub, which are connected by slender beams and store torsion energy in beam bending. In a preferred embodiment, the beams are fixed to the outer hub on one end and attached to the inner hub by a pin and slot on the other. The spring is capable of deflecting at least ±

$$\frac{π}{6}$$

radians, higher than any existing planar torsion spring designs, and is capable of providing 100 N·m of torque.

With this form factor, the planar spring design provides a more compact alternative to elastic elements currently used in series elastic actuators. In addition, using the models presented, the design dimensions, material, and slot geometry of the planar torsion spring can be parameterized to design springs that meet specific requirements for different applications. In addition to quantifying performance, the models provide the foundation for further weight, efficiency, and performance optimization.

The objective of a preferred embodiment of the invention is to provide a compact, torsionally compliant element for an SEA that can be used in the knee joint of an exoskeleton design. This particular application results in three main functional requirements that the design will preferably satisfy: torque response of 100 N·m, deflection of ±

$$\frac{π}{6}$$

radians, and minimization of the design's size and mass. In addition to the biomechanical functional requirements, wearable robotics require a compact form factor that is comfortable to wear and does not disturb the natural movement of users. The ideal torsion spring for this application is therefore one that is able to provide biologically appropriate deflections and torques while minimizing width, diameter, and mass.

In order to be able to properly evaluate the developed spring design, several additional physical constraints were applied to the design of the new spring in order to make comparative analysis more analogous. The design was compared to the NASA planar torsion spring, the current configuration of which is capable of deflecting up to ±

$$\frac{π}{36}$$

radians, has a maximum diameter of 0.085 meters and a maximum width (planar thickness) of 0.0005 meters, and is made from maraging steel.

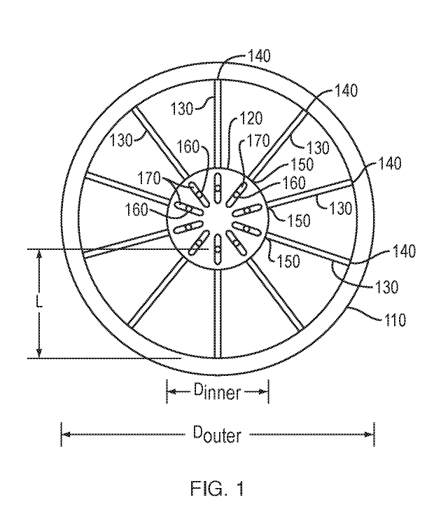

As shown in FIG. 1, a planar torsion spring according to a preferred embodiment of the invention is a disk shape spring that consists of two concentric hubs, outer hub 110 and inner hub 120. Hubs 110, 120 are connected by beams 130 that undergo bending to provide torsional compliance (angular deflection). On one end 140, each beam 130 is fixed to outer hub 110, and on the other end 150, each beam 130 is constrained to inner hub 120 through a pin 160 and slot 170. As outer hub 110 rotates relative to inner hub 120, beams 130 bend and pins 160 move along respective slots 170.

FIG. 1 depicts a preferred design, a 10-beamed, straight slotted torsion spring. In a preferred embodiment of this design, Dₒᵤₜₑᵣ=0.112 meters, Dᵢₙₙₑᵣ=0.05 meters, and L=0.035 meters. The resulting maraging steel torsion spring has a mass of 98 grams, outer diameter of 0.112 meters, and width of 0.005 meters. The spring uses slender beams which have a length of 0.035 meters, height of 0.001 meters, and width of 0.005 meters. The resulting spring design is capable of rotating ±

$$\frac{π}{6} .$$

This max angular rotation is 6 times that of the NASA planar torsion spring, which has a slightly smaller diameter of 0.085 meters.

While having a pin, straight-slot constraint on the inner hub has the disadvantage of friction forces and efficiency losses, it allows the spring to undergo much higher angular deflections than existing planar torsion springs, which fix the beams on both the inner and outer hub. In the torsion spring of the invention, the beams that undergo bending to provide the angular deflection are fixed onto the outer hub on one end, but are constrained using a pin and slot to the inner hub on the other. Torsional compliance is provided as the outer hub rotates with respect to the inner hub, bending the slender beams.

In addition to allowing for higher deflections, the pinned, slotted design also allows for various parameters of the spring to be customized to meet different requirements for specific applications. In the compact design of a planar torsion spring, the spring thickness can be adjusted to obtain the desired stiffness and maximum torque. A fully parametrized model can be developed for each application such that the effects of material, beam width, beam thickness, and slot design on efficiency, torque response and deflection are understood. Such a model can help optimize the spring size, weight, max stresses, and stiffness for the specific application. Furthermore, this novel design opens up an entire design space with potential optimization and performance trade-offs that fixed, fixed beam torsion springs lack. The main advantage of the preferred spring design is the ability to undergo comparatively higher angular deflections.

For applications in prosthesis and exoskeletons, efficiency is also of importance and alternative features, such as using a bearing at the pin-slot interface or using another method of providing rolling contact to reduce frictional losses can also be advantageously employed. Similarly, design features can be altered to further minimize the mass and size of the spring. In one embodiment, the shape of the beams are altered to make more efficient use of the mass by equalizing the stress along the surface of the beam, where the max stress occurs for beam bending.

Multiple prototypes have been constructed in the lab. Aluminum prototypes were developed with the waterjet, and plastic versions were printed with a 3D printer. The prototypes have all been about 100 mm×100 mm×10 mm. These springs have shown that the basic concept of using separate pieces to allow for different beam end conditions results in planar torsional springs that can undergo greater deflections in a smaller and lighter package. These prototypes have also shown that it is possible to mechanically program the stiffness characteristics of the springs to achieve variable spring rates.

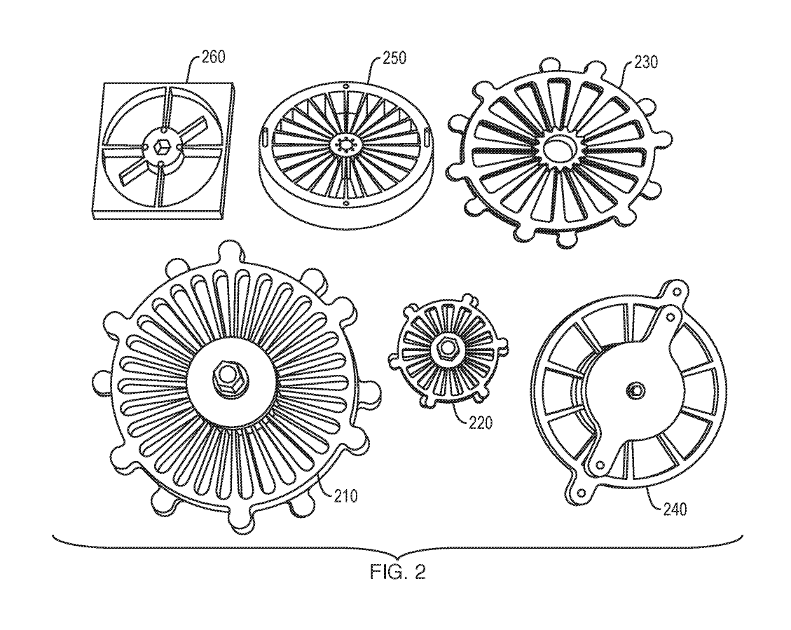

FIG. 2 depicts several embodiments of the various prototypes of the torsional spring. Prototypes 210, 220, 230, 240, 250 were all printed, while prototype 260 is made of 7075 Aluminum. These prototypes demonstrate different implementations of the torsional spring design according to the present invention.

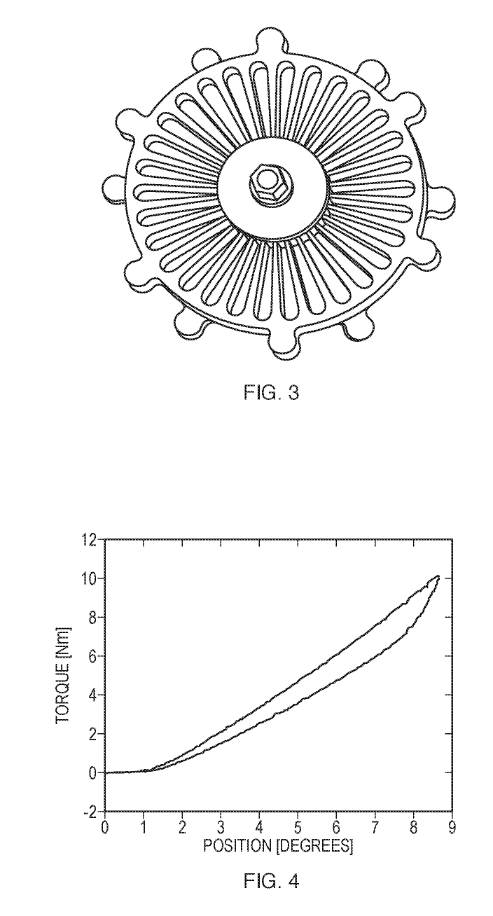

The prototype depicted in FIG. 3 includes two torsional springs in series in order to achieve maximum deflections of +/−20 degrees. This spring was tested with a torque sensor and rotation sensor in order to measure the stiffness and hysteresis, which are graphed in FIG. 4. It is believed that the hysteresis shown in FIG. 4 is due mostly to the material properties of the ABS and is therefore not inherent to the design.

Design Parameters and Mathematical Models.

The various approaches that were taken in attempting to find the optimal torsion spring design for prosthesis and exoskeleton applications are presented below, along with the mathematical models that were developed to understand the effects of design parameters and analyze spring performance. These models provide the fundamentals required to further parametrize and optimize the torsion spring design for specific applications. It will therefore be clear to one of skill in the art that these approaches and models can be used to design springs that meet the specific requirements and design constraints of applications other than the ones described herein.

Mechanical Energy Storage.

There are two types of mechanical energy storage in materials: hydrostatic energy and shear energy. In designing a compact spring, it is extremely difficult to apply hydrostatic forces and appropriately constrain the material. The first design approach was to minimize the amount of material in a spring while maximizing energy storage. However, due to the differences in types of loading, both of which result in material shear energy storage, the final design approach focused on minimizing stiffness in loading to maximize deflection rather than maximizing energy storage.

Energy storage density of different materials. The Von Mises Yield Criterion helps provide an understanding of how materials store energy and how materials yield. In the derivation of the Von Mises Yield Criterion, a material yields due to maximum shear energy. Since the Von Mises stress is calculated from distortion energy, or the amount of shear energy before failure, hydrostatic energy is disregarded. Therefore, it is extremely mechanically difficult, but theoretically possible, to store incredibly large amounts of energy in a material through hydrostatic forces. Any stress states with the same distortion energy will have the same Von Mises stress, and the material fails when the Von Mises stress exceeds the yield strength of the material.

In exploring the max energy storage, the amount of energy stored before failure in different materials was explored. The approximate modulus of resilience, which is the maximum energy that can be absorbed per unit volume without creating permanent distortions, was calculated by

$${U}_{r} = \frac{{σ}_{y}^{2}}{2 E} \qquad ( 2.1 )$$

where σ_{y} is the yield stress and E is the Young's Modulus. In using Equation 2.1, the Young's Modulus is assumed to be linear, and therefore the equation is only accurate as an approximation for materials such as rubber, which have a non-linear Young's modulus.

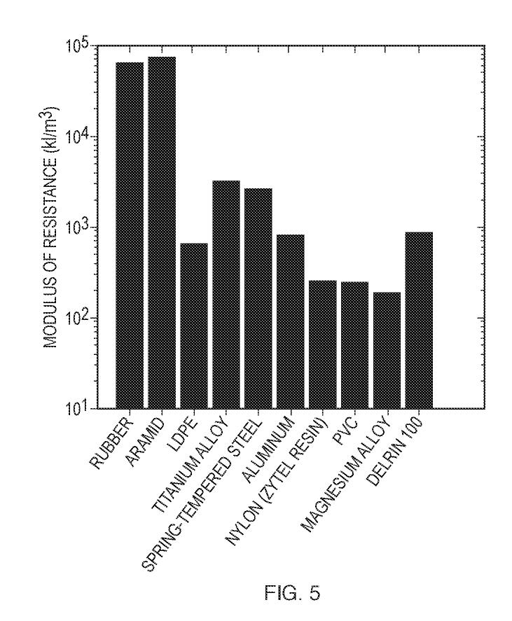

In calculating the modulus or resistance of materials, the amount of energy that a material can store before it fails can be compared. In FIG. 5, the modulus of resistance for various materials are presented and compared. From FIG. 5, it can be seen that traditional materials such as spring-tempered steel or even a titanium alloy can only store a tenth of the amount of energy per unit volume that materials such as aramid or rubber can. However, it should be understood that the modulus of resilience calculates the tensile energy stored before failing and is therefore a poor estimation of maximum shear energy for non-isoptropic materials, such as, but not limited to, aramid, which fail at much lower stresses in other loading conditions

Beam Bending vs. Axial Loading. Because hydrostatic loading on a material is extremely difficult to implement, springs store shear energy. To this end, there are two main types of loads to store energy: axial loading and beam bending. In most existing planar torsion springs, beams, which are fixed to an inner hub at one end and an outer hub on the other, provide energy storage through bending. In designing a spring for this particular application, high deflections are desirable, and therefore stiffness needs to be minimized. For equivalent axial loading and bending loads on identical beams, the beam undergoing bending sees higher deflections. The analysis and comparison of these two types of loading on a simple beam is as follows:

For axial loading:

$$F = \frac{EA}{L} δ \qquad ( 2.2 )$$

where F is the load force, A is the beam cross sectional area, L is the beam length, and δ is the beam deflection at the end. From this, the stiffness is

$${k}_{axial} = \frac{EA}{L} \qquad ( 2.3 )$$

For beam bending:

$$F = \frac{3 EI}{{L}^{3}} δ \qquad ( 2.4 )$$

where I is the second moment of area of a rectangular beam

$$I = \frac{{bh}^{3}}{12} \qquad ( 2.5 )$$

in which b is the width and h is the height of the beam. The stiffness is defined by

$${K}_{bend} = \frac{3 EI}{{L}^{3}} \qquad ( 2.6 )$$

In the case where the beams have an L=0.035 meters, b=0.005 meters, and h=0.001 meters, the bending stiffness is approximately 4000 times less than that of the axial stiffness. Because deflection is directly proportional to force in both beam bending and axial loading, the lower bending stiffness will result in much higher deflections at equivalent loads. Since high deflections are desired, the design approached storing torsion energy through beam bending. The modeling of such a spring design's performance was based on derivations using the EulerBernoulli beam theory [Roy R. Craig. Mechanics of Materials. Wiley, 2011].

Beam Modeling and Analysis.

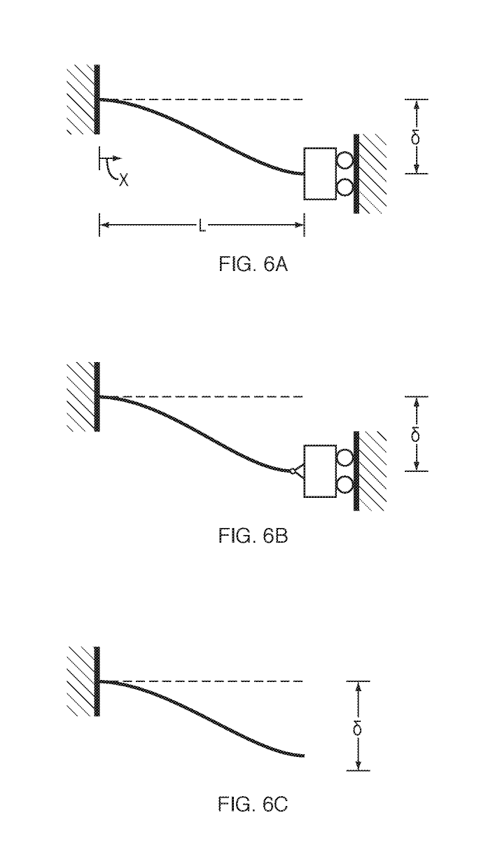

Beam bending and boundary conditions. In pursuing a planar torsion spring design in which the beams store energy in beam bending, mathematical modeling of beam bending is utilized to best determine beam boundary conditions that would maximize deflection before yielding. The three beam bending boundary conditions explored are shown in FIGS. 6A-C. For each of the beam boundary conditions, deflection profiles, max stresses, energy stored, and stiffnesses are modeled. Shown in FIG. 6A is a Fixed, Fixed-Roller beam, in FIG. 6B depicts a Fixed, Pin-Roller beam, and FIG. 6C depicts a Fixed, Free beam.

In order to provide analogous comparison between different beam conditions, all beams have the listed properties. The dimensions of the beam used in the models are the same as those of the final tested spring design. These parameters are: Dimensions: Length: 0.035 meters; Width: 0.005 meters; and Height: 0.001 meters; Material: Maraging Steel; Young's Modulus: 210×109 Pascals; Yield Stress: 2.0×109 Pascals; and Ultimate Yield Stress: 3.5×109 Pascals.

Fixed, Fixed-Roller Beam.

The case in which the beam is fixed on one end and fixed-roller on the other is shown in FIG. 6A. For existing planar springs that use beam spokes, the fixed, fixed-roller boundary condition approximates the loading and stress characteristics. It is necessary to first derive the equations that describe the beam deflection, beam slope, and beam bending moment. The amount of energy stored in bending and in tension are then calculated, from which stiffness can be found and compared.

In order to model the system, the deflection profile of the beam is first derived using the generalized equation for neutral axis deflection with respect to x

w(x)=Ax³+Bx²+Cx+D (3.1)

where x is the position along the length of the beam and A, B, C, and D are constants that are dependent on the end conditions of the beam [Roy R. Craig. Mechanics of Materials. Wiley, 2011]. The derivative of the beam deflection equation

\dot{w}(x)=3Ax²+2Bx+C (3.2)

gives the slope of the beam as a function of position along the length. The second derivative of beam deflection is proportional to the bending moment along the length of the beam.

\ddot{w}(x)=6Ax+2B (3.3)

From these three generalized equations, the following boundary conditions can be applied for a beam undergoing a bending deflection of δ:

w(0)=\dot{w}(0)=0 (3.4)

due to the fixed condition at x=0 and

w(L)=δ;\dot{w}(L)=0 (3.5)

due to the fixed, roller condition at L=0. From the boundary conditions, the generalized constants can be solved and substituted for equations (3.1), (3.2), and (3.3).

$$\begin{matrix}w ( x ) = \frac{- 2 δ}{{L}^{3}} {x}^{3} + \frac{3 δ}{{L}^{2}} {x}^{2} & ( 3.6 ) \\ \dot{w} ( x ) = \frac{- 6 δ}{{L}^{3}} {x}^{2} + \frac{6 δ}{{L}^{2}} x & ( 3.7 ) \\ \ddot{w} ( x ) = \frac{- 12 δ}{{L}^{3}} x + \frac{6 δ}{{L}^{2}} & ( 3.8 )\end{matrix}$$

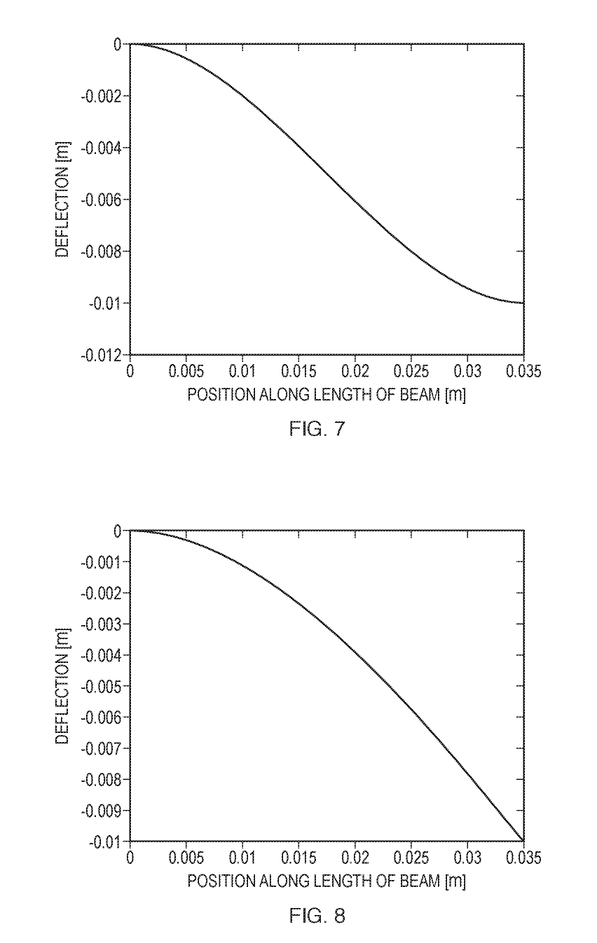

With the generalized constants solved in terms of δ, Equation (3.6) can be plotted with the beam undergoing 0.01 meters of deflection. FIG. 7 is a graph of the deflection profile of a fixed, fixed-roller beam undergoing 0.01 meters of deflection.

Due to the fixed condition at each end of the beam, there is an inflection point at x=L/2 where the change in slope of the beam is zero. The fixed condition and fixed distance between the ends of the beam make it such that as the beam deflects, the elongation of the beam due to bending increases the axial loading of the beam at high deflections. The equation for the elongated beam length is

S=∫₀^{L}\sqrt{1+\dot{w}(x)²}dx (3.9)

where \dot{w}(x) is the slope of the beam as a function of distance along the length solved in Equation 3.7 [Roy R. Craig. Mechanics of Materials. Wiley, 2011]. The resulting elongation of the beam will be used to calculate and compare the stiffnesses and stresses of the different beams.

Fixed, Pinned-Roller Beam.

In modeling the fixed, pinned-roller beam shown in FIG. 6B, a similar approach was taken. In this case, while the boundary conditions due to the fixed end at x=0 is the same as the fixed, fixed-roller beam,

w(0)=\dot{w}(0)=0 (3.10)

the boundary conditions at x=L are

w(L)=δ;\dot{w}(L)=0 (3.11)

due to the pin. These boundary conditions, when used to solve for the generalized constants result in the following equations where

$$w ( x ) = \frac{- δ}{2 {L}^{3}} {x}^{3} + \frac{3 δ}{2 {L}^{2}} {x}^{2} \qquad ( 3.12 )$$

describes the deflection as a function of position along the length of the beam,

$$\dot{w} ( x ) = \frac{- 3 δ}{2 {L}^{3}} {x}^{2} + \frac{3 δ}{{L}^{2}} x \qquad ( 3.13 )$$

describes the slope of the beam, and

$$\ddot{w} ( x ) = \frac{- 3 δ}{{L}^{3}} x + \frac{3 δ}{{L}^{2}} \qquad ( 3.14 )$$

describes the bending moment in the beam for a specific deflection, δ.

The deflection profile of a fixed, pinned-roller beam undergoing 0.01 meters of deflection can be seen in FIG. 8.

It should be understood that, due to the boundary conditions at the pinned end, w(L)=δ; \dot{w}(L)=0, the deflection profile of the fixed, pinned-roller beam is identical to that of the fixed, free cantilever beam. However, unlike the fixed-free cantilever beam, the fixed distance between the fixed end and the pinned end result in an increase in axial stresses in the beam at high deflections. Similar to the fixed, fixed-roller beam, the equation for beam elongation is given by

S=∫₀^{L}\sqrt{1+\dot{w}(x)²}dx (3.15)

where the different boundary conditions of the fixed, pinned-roller beam result in a different w(x), solved in Equation 3.13.

Fixed, Free Beam.

In the fixed, free beam (FIG. 6C), which is more commonly referred to as a cantilever beam, the boundary conditions are the same as that of the fixed, pin-roller beam.

w(0)=\dot{w}(0)=0 (3.16)

w(L)=δ;\dot{w}(L)=0 (3.17)

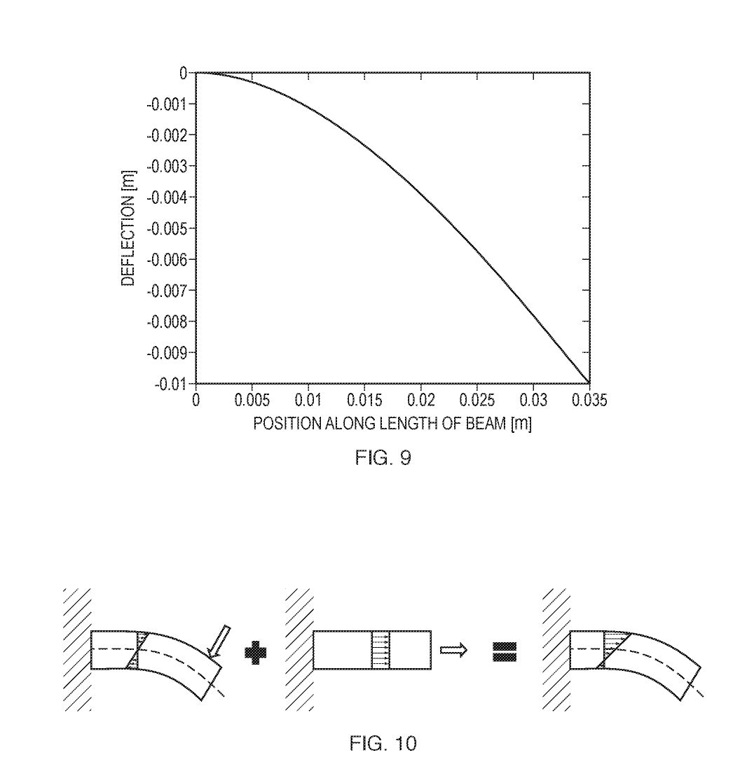

This results in the following equations and an identical beam deflection profile, shown in FIG. 9 which is the deflection profile of a fixed, free cantilever beam undergoing 0.01 meters of deflection.

$$\begin{matrix}w ( x ) = \frac{- δ}{2 {L}^{3}} {x}^{3} + \frac{3 δ}{2 {L}^{2}} {x}^{2} & ( 3.18 ) \\ \dot{w} ( x ) = \frac{- 3 δ}{2 {L}^{3}} {x}^{2} + \frac{3 δ}{{L}^{2}} x & ( 3.19 ) \\ \ddot{w} ( x ) = \frac{- 3 δ}{{L}^{3}} x + \frac{3 δ}{{L}^{2}} & ( 3.20 )\end{matrix}$$

As modeled, the fixed, free beam is identical to the fixed, pinned-roller beam in deflection profile, beam slope, and beam bending moments. However, it should be understood that, in the case of the cantilever beam, the axial elongation is zero and does not affect the stresses in the beam.

Beam Stresses and Stiffness Comparison.

From the equations w(x), \dot{w}(x), and \ddot{w}(x) for each beam, analysis on the amount of stress, bending energy, and tensile energy in each beam undergoing 0.01 meters of deflection can be performed.

Maximum Beam Stresses. For the cases in which the beam is undergoing both bending and tensile loading, superposition of the axial and bending stresses in the beam can be applied to calculate the resulting maximum stress in each beam condition. As shown in FIG. 10, the maximum stresses will occur on the top surface of the loaded beam. In the case of the fixed, fixed-roller beam, and the fixed, pinned-roller beam, the maximum stress is equal to the sum of the bending stress and tensile stress. The tensile stress results from the elongation of the beam as it undergoes bending.

As shown in FIG. 10,

$$\begin{matrix}{σ}_{total} = {σ}_{bend} + {σ}_{axial} \\ where & ( 3.21 ) \\ {σ}_{bend} ( x , y ) = \frac{M ( x ) y}{I} ; {σ}_{axial} = E ε & ( 3.22 )\end{matrix}$$

in which x is the distance along the beam, y is the distance from the neutral axis, and E is the axial strain [Roy R. Craig. Mechanics of Materials. Wiley, 2011]. The bending stresses in each of the three beams is defined as:

M(x)=−EI\ddot{w}(x) (3.23)

in which E is the Young's Modulus of maraging steel, I is the area moment of inertia of a rectangular cross section, and \ddot{w}(x) were solved for each beam in Equations 3.8, 3.14, and 3.20 [Roy R. Craig. Mechanics of Materials. Wiley, 2011].

The max bending stress occurs at

$$x = 0 ; y = \frac{h}{2} \qquad ( 3.24 )$$

for all beam cases, resulting in

$${σ}_{bend} = \frac{M ( 0 ) \frac{h}{2}}{I} \qquad ( 3.25 )$$

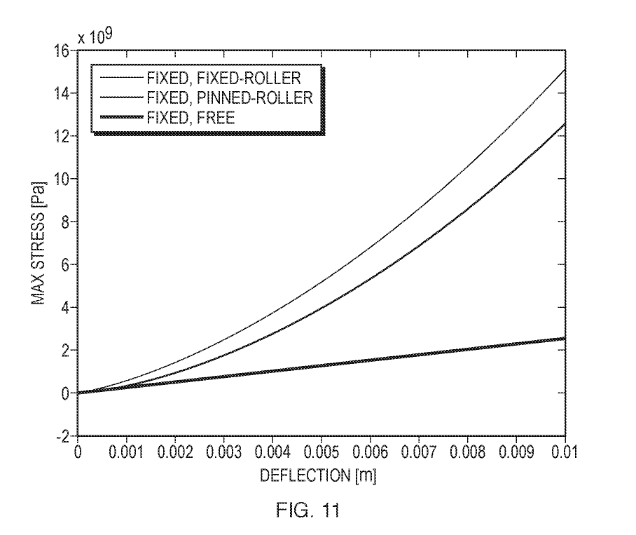

In addition to bending stresses, the fixed, fixed-roller, and fixed, pinned-roller beams also undergo axial stresses at higher deflections due to the elongation of the beam. The resulting axial stress is defined as:

$${σ}_{axial} = \frac{E ( S - L )}{L} \qquad ( 3.26 )$$

where S was solved for in Equations 3.9 and 3.15 for the fixed, fixed-roller and fix, pinned-roller beams, respectively. Using the superposition of stresses, the total max stress of each beam undergoing 0.01 meters of deflection calculated as a function of deflection is plotted in FIG. 11.

From this comparison, it can be seen that at very small deflections, all beams increase in stress very similarly. However, as the deflection increases, the tensile stresses begin to dominate, and the fixed, fixed-roller beam and fixed, pinned-roller beam begin to see much higher maximum stresses. The rate of max stress increase is higher for the beams with more constraints at x=L.

The fixed, pinned-roller beam, while having the same deflection profile, begins seeing higher max stresses at high deflections. As expected, the max stresses of the fixed, pinned-roller beam is equal to that of the fixed, free beam for higher deflections than the fixed, fixed-roller beam.

Beam Stiffnesses. While the max stresses provide valuable insight into the beams as they undergo deflection, it is important to understand the stiffness of each beam and how it changes with deflection. The stiffness of each beam was calculated by taking the numerical derivative of the energy stored in each beam. First, the total amount of energy stored in a beam as a function of deflection was calculated.

$$\begin{matrix}{U}_{total} ( δ ) = {U}_{bend} ( δ ) + {U}_{axial} ( δ ) & ( 3.27 ) \\ {U}_{bend} ( δ ) = \frac{EI}{2} {\int }_{0}^{L} {\ddot{w} ( x )}^{2} dx & ( 3.28 )\end{matrix}$$

where \ddot{w}(x) is defined by Equations 3.8, 3.14, and 3.20 for the fixed, fixed-roller beam; fixed, pinned-roller beam; and fixed, free beam, respectively. Additionally, in the case of the fixed, fixed-roller beam and the fixed, pinned-roller beam, tensile energy is defined by

$${U}_{axial} ( δ ) = \frac{AE}{2} {\int }_{0}^{L} {∈}^{2} dx \qquad ( 3.29 )$$

After calculating the total amount of energy stored in each beam for 0<δ<0.01 meters, the numerical derivatives were taken.

$$\begin{matrix}\frac{{dU}_{total}}{d δ} = F ( δ ) & ( 3.30 ) \\ \frac{{d}^{2} {U}_{total}}{d {δ}^{2}} = k ( δ ) & ( 3.31 )\end{matrix}$$

where k(δ) is the stiffness of the beam as a function of deflection.

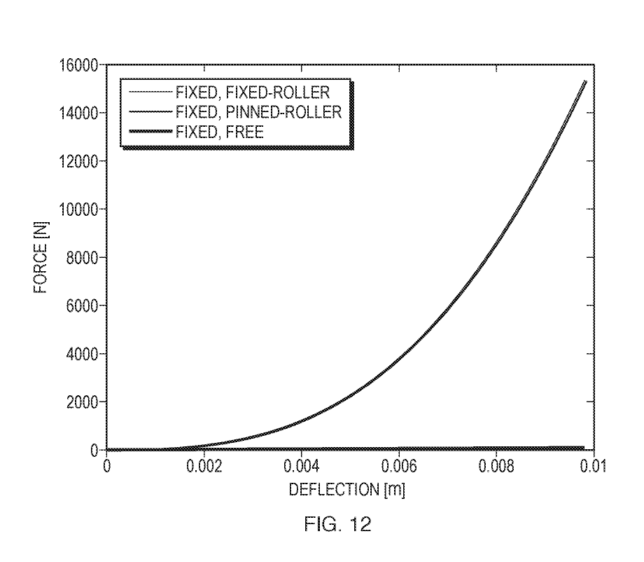

By plotting the beam force as a function of deflection, as shown in FIG. 12, it can be seen that the fixed, fixed-roller beam and fixed, pinned-roller beam forces begin to increase drastically at higher deflections. All three beams provide the same force when undergoing small deflections. However, as the fixed-roller and pinned-roller beams begin to undergo axial strain at higher deflections, the forces begin to differ drastically from that of the fixed-free beam which is undergoing pure bending.

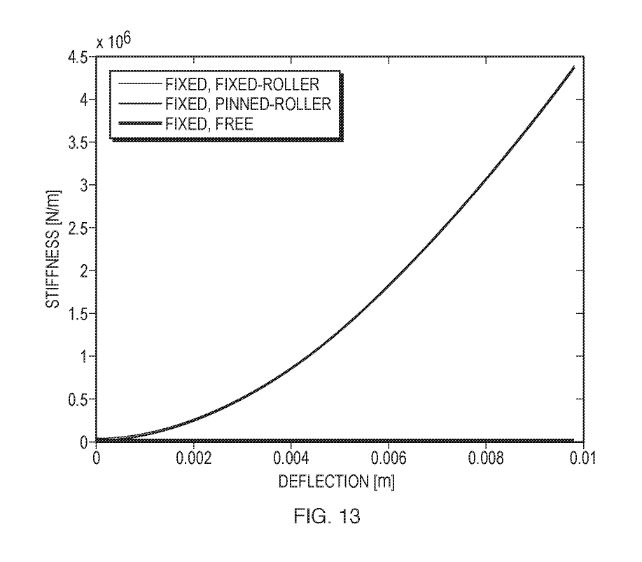

The effect of axial loading does not become significant until approximately 0.002 meters of deflection. In FIG. 11, this is also the deflection at which the max stresses of the three beams begin to diverge. However, from FIG. 13, which is a graph of the stiffness of each of the three beams as a function of deflection, the axial loading's effect on the stiffnesses of the beams is apparent at much lower deflections than 0.002 meters.

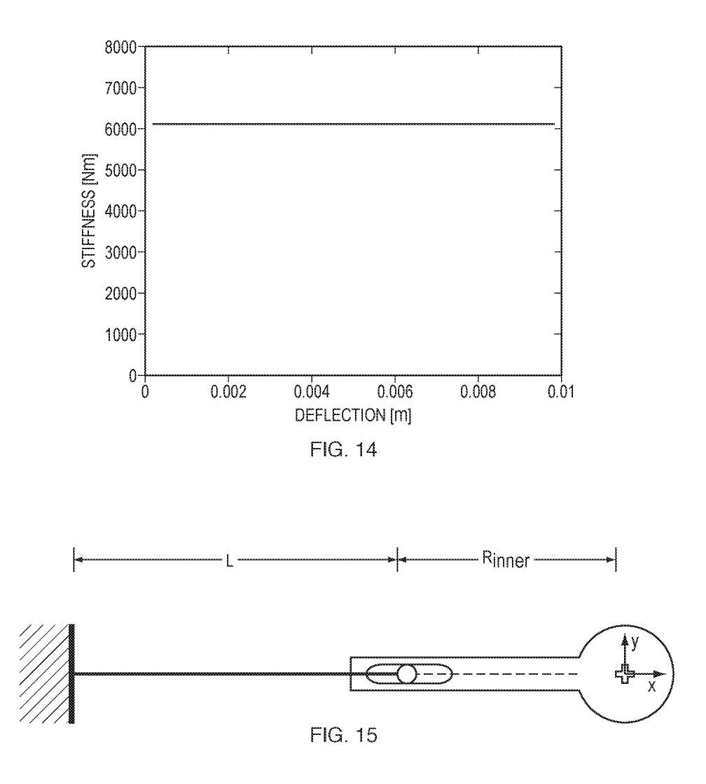

FIG. 14 shows that the stiffness of the fixed, free cantilever beam is constant, as expected as it is independent of deflection. Additionally, the stiffnesses of the fixed, free beam are up to 3 orders of magnitude less than that of the other two beams.

Spring Modeling

In designing a planar torsion spring that is capable of large angular deflections, it is desirable that the beams bending to store the torsional energy be as close to the fixed, free beam condition as possible. From analyzing the various beam bending conditions, such a beam configuration is desired to decrease stiffness, especially at high deflections. In pursuing such a design, a fixed, pinned-slotted beam design was explored, the first of which had the end of the beam following a straight, radial slot as the beam deflects (FIG. 15). After exploring the efficiency and torque performance of this pinned, straight-slotted beam design, a more complex curved slot design was modeled and analyzed.

Pinned, Straight-Slot Constrained Beam.

As shown in FIG. 15, which is a schematic of a beam and inner hub of the first fixed, pinned-slotted beam design, the slot is straight and allows the pinned beam end to move radially as the inner hub of the spring turns.

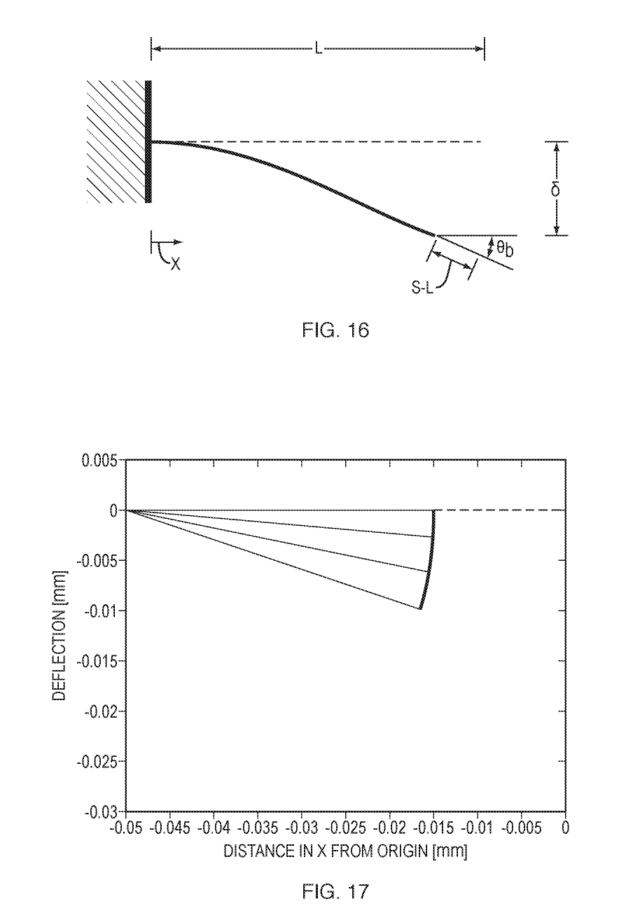

Beam End Trajectory. In order to model the beam bending and forces on the pin, the trajectory of the beam end of an unconstrained cantilever beam was first calculated. In calculating this trajectory, it is assumed that the force required for deflection is applied at the tip of the beam and the force is always perpendicular to the changing neutral axis of the beam. FIG. 16 depicts the trajectory of the beam tip as it bends, being calculated as a function of beam tip angle, deflection, and beam elongation due to bending.

The trajectory of the beam tip is calculated and plotted with the origin at the hub. In this calculation, the x and y-component of the end trajectory is calculated to be

x=Rᵢₙₙₑᵣ−((S−L)cos(θ_{b})) (4.1)

y=δ (4.2)

where Rᵢₙₙₑᵣ=0.015 meters and S is the projected elongated length of a constrained beam undergoing bending. It is to be understood that the cantilever beam is not undergoing elongation because the beam is unconstrained at x=L.

S=∫₀^{L}\sqrt{1+\dot{w}(x)²}dx (4.3)

where θ_{b} is the calculated beam angle with respect to the neutral axis at x=L

θ_{b}=arctan(\dot{w}(x)) (4.4)

FIG. 17 is plot of the trajectory of the beam tip undergoing 0.01 meters of deflection. In calculating the trajectory, the origin is set at the center of the inner hub.

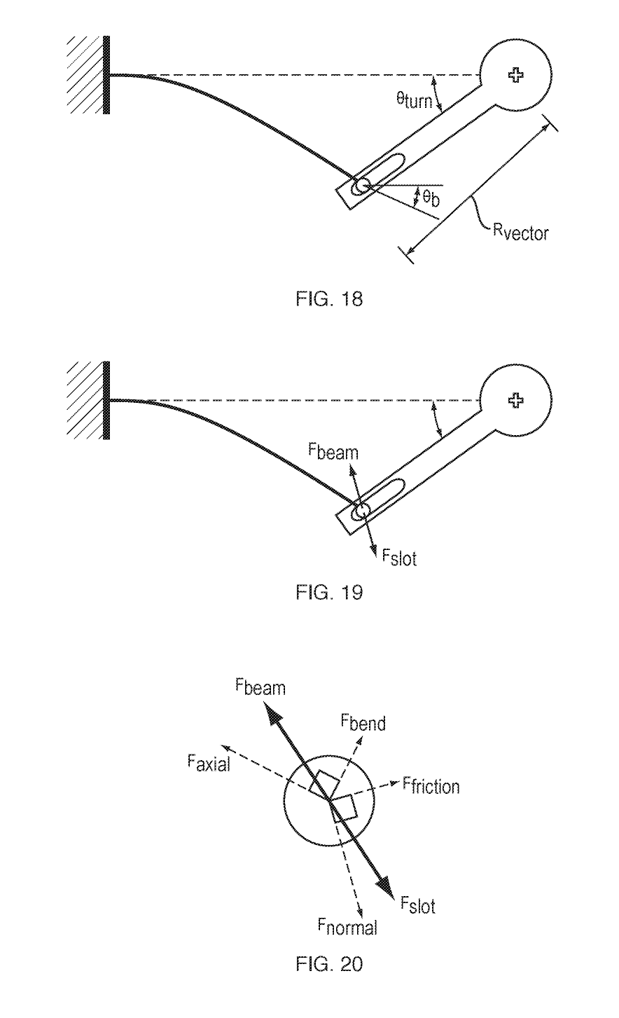

Beam and Slot Forces. In order to understand the torque response of this pinned, slotted beam design, the forces acting on the pin must be calculated. It is to be understood that, as the inner radius turns and deflects the beam, the effective radius on which the forces act changes. FIG. 18 shows that, as the inner hub undergoes angular deflection, the resulting beam deflection changes the radius vector on which the torque is acting.

The deflected beam trajectories in Equations 4.1 and 4.2 were calculated with respect to the hub center as origin, and therefore are the x and y components of \vec{R}_{vector}.

Rₓ=Rᵢₙₙₑᵣ((S−L)cos(θ_{b}));R_{y}=−δ (4.5)

From this, the θₜᵤᵣₙ can be calculated.

$${θ}_{turn} = arctan ( \frac{{R}_{y}}{{R}_{x}} ) \qquad ( 4.6 )$$

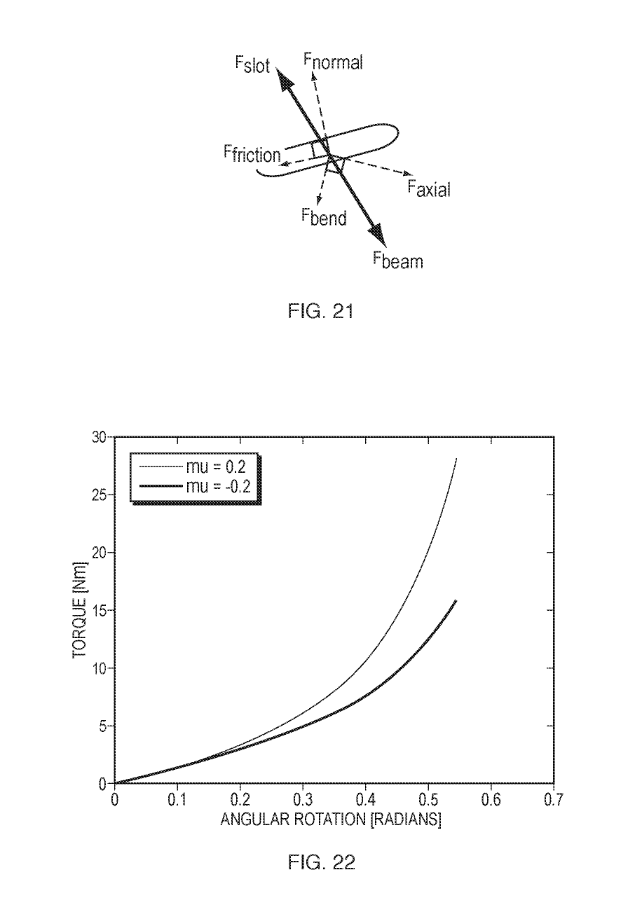

FIG. 19 depicts the two forces that act on the pin at the tip of the beam. FIG. 20 depicts the forces due to each body acting on the pin, and also shows the forces on the pin decomposed into their respective parts. FIG. 21 depicts the forces acting on the slot, and also shows the forces on the slot decomposed into their respective parts.

\vec{F}_{beam} and \vec{F}ₛₗₒₜ are both vectors that are dependent on θₜᵤᵣₙ. \vec{F}_{beam} is a result of the beam bending force and axial force. \vec{F}ₛₗₒₜ is a result of the friction force that acts on the pin, which acts along the slot, and the force that acts normal to the slot. The pin was modeled as having a zero diameter.

\vec{F}_{beam}+\vec{F}ₛₗₒₜ=0 (4.7)

Of these forces, both the direction and magnitude of \vec{F}_{bend} is known. For \vec{F}ₐₓᵢₐₗ, direction is known, but magnitude is unknown. Similarly, only the directions are known for both \vec{F}_{friction} and \vec{F}ₙₒᵣₘₐₗ. In order to characterize the torque response of the beam, \vec{F}ₛₗₒₜ as a function of δ is required. From Equation 4.7 and what is known about the direction of the forces, the following equation is derived:

$$\vert {\vec{F}}_{axial} \vert [ \begin{matrix}- {\hat{F}}_{bend - y} \\ {\hat{F}}_{bend - x}\end{matrix} ] + \vert {\vec{F}}_{slot} \vert [ \begin{matrix}{\hat{F}}_{slot - x} \\ {\hat{F}}_{slot - y}\end{matrix} ] + \vert {\vec{F}}_{bend} \vert [ \begin{matrix}{\hat{F}}_{bend - x} \\ {\hat{F}}_{bend - y}\end{matrix} ] = 0 \qquad ( 4.8 )$$

where the unit vectors of \vec{F}ₛₗₒₜ are

$$\begin{matrix}{\hat{F}}_{slot - x} = \vert {\vec{F}}_{normal} \vert {\hat{R}}_{y} + μ \vert {\vec{F}}_{normal} \vert {\hat{R}}_{x} & ( 4.9 ) \\ {\hat{F}}_{slot - y} = - \vert {\vec{F}}_{normal} \vert {\hat{R}}_{x} + μ \vert {\vec{F}}_{normal} \vert {\hat{R}}_{y} & ( 4.10 ) \\ \vert {\vec{F}}_{bend} \vert = 3 EI \frac{δ}{{L}^{3}} & ( 4.11 )\end{matrix}$$

Substituting this into and rearranging Equation 4.8,

$$\begin{matrix}[ \begin{matrix}- {\hat{F}}_{bend - y} & \vert {\vec{F}}_{normal} \vert {\hat{R}}_{y} + μ \vert {\vec{F}}_{normal} \vert {\hat{R}}_{x} \\ {\hat{F}}_{bend - x} & - \vert {\vec{F}}_{normal} \vert {\hat{R}}_{x} + μ \vert {\vec{F}}_{normal} \vert {\hat{R}}_{y}\end{matrix} ] [ \begin{matrix}\vert {\vec{F}}_{axial} \vert \\ \vert {\vec{F}}_{slot} \vert\end{matrix} ] = - \vert {\vec{F}}_{bend} \vert [ \begin{matrix}{\hat{F}}_{bend - x} \\ {\hat{F}}_{bend - y}\end{matrix} ] & ( 4.12 ) \\ and & \\ [ \begin{matrix}\vert {\vec{F}}_{axial} \vert \\ \vert {\vec{F}}_{slot} \vert\end{matrix} ] = {[ \begin{matrix}- {\hat{F}}_{bend - y} & \vert {\vec{F}}_{normal} \vert {\hat{R}}_{y} + μ \vert {\vec{F}}_{normal} \vert {\hat{R}}_{x} \\ {\hat{F}}_{bend - x} & - \vert {\vec{F}}_{normal} \vert {\hat{R}}_{x} + μ \vert {\vec{F}}_{normal} \vert {\hat{R}}_{y}\end{matrix} ]}^{- 1} ( - \vert {\vec{F}}_{bend} \vert ) [ \begin{matrix}{\hat{F}}_{bend - x} \\ {\hat{F}}_{bend - y}\end{matrix} ] & ( 4.13 )\end{matrix}$$

From Equation 4.13, the magnitudes of \vec{F}ₐₓᵢₐₗ and \vec{F}ₛₗₒₜ are calculated, where μ is the coefficient of friction between the pin and the slot. Using this, the entirety of \vec{F}ₛₗₒₜ vector can be calculated for all 6.

\vec{F}ₛₗₒₜ=−\vec{F}_{bend}−\vec{F}ₐₓᵢₐₗ (4.14)

Torque and Efficiency. From the \vec{F}ₛₗₒₜ calculated in Equation 4.14, the torque resulting from a single pinned, slotted beam is

\vec{r}=\vec{R}_{vector}×\vec{F}ₛₗₒₜ (4.15)

In an example case, μ=0.2, which is the coefficient of friction for lubricated steel-on-steel contact [Erik Oberg, Franklin D. Jones, Holbrook L. Horton, and Henry H. Ryffel. Machinery's Handbook 29th Edition. Industrial Press, 2012]. In order to simulate angular deflection in the opposite direction, μ=−0.2 is used. Assuming that the torsion spring design has 10 beams, all acting in parallel, the torque response of one planar torsion spring for turning the spring and then returning it to equilibrium is shown in FIG. 22.

In plotting the torque response, the effect of hardening can be observed. The stiffness of the beams increase as the beams begin to see tensile stresses at higher deflections. Also, as expected, the torque response for μ=0.2 is higher than that of μ=−0.2. When deflecting the beams in one direction, the effect of friction on the torque is additive, while in reversing the deflection, the effect is subtractive.

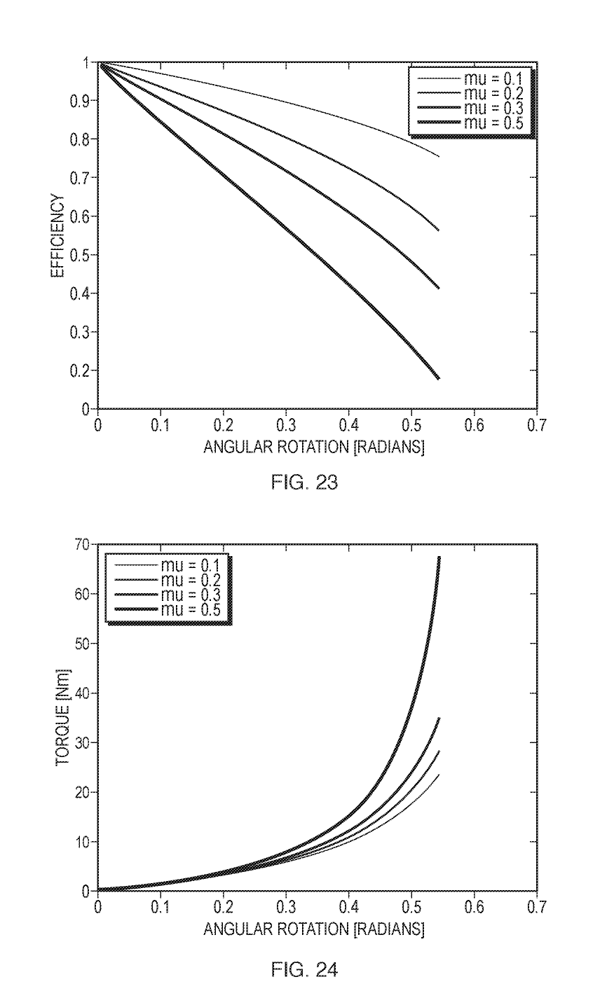

From the data presented in FIG. 22, the efficiency of the spring as a function of deflection can also be calculated and plotted. FIG. 23 depicts the efficiency of the spring as a function of angular rotation of the inner hub for various coefficients of friction, μ. This efficiency is calculated by taking the ratio of torque resulting from negative μ to torque resulting from positive μ at each deflection.

It is demonstrated that efficiency is highly dependent on μ, with lower efficiencies seen at higher μ. If the spring is being designed for applications in which high efficiency is desired, lubrication and pin material are extremely important. However, as seen in FIG. 24, which is a plot of the torque response of a 10-beam spring for various coefficients of friction, higher μ allow for higher torque responses, at the cost of efficiency, especially at high deflections. Depending on the application of the torsion spring, these parameters can be optimized to obtain the desired spring characteristics, whether it be high torque response or high efficiency.

Maximum Stress. In order to estimate the maximum stress in the beam, Equation 3.25 is used. At a maximum angular deflection of ±

$$\frac{π}{6}$$

radians, the max stress in the cantilever beam is 2.4 GPa. For maraging steel, σᵤₗₜ=3.5 GPa.

It should be understood that, while the pinned, slotted beam used in this spring design mimics the behavior of a cantilever beam, there are axial stresses in the beam that are not estimated by this simple estimation. Therefore, it should be expected that max stresses be higher in the actual spring spokes. In order to decrease the max stress in a beam, the equation for moment about the neutral axis, which was solved in Equation 3.23, can be explored. It can be seen that, M(x) and in turn, the max stress can be decreased as L is increased. This has a quadratic effect on the max stress in the bending cantilever beam. Furthermore, a variable cross-sectional area beam can be explored to further decrease stiffness and mass.

Curved Slot Design.

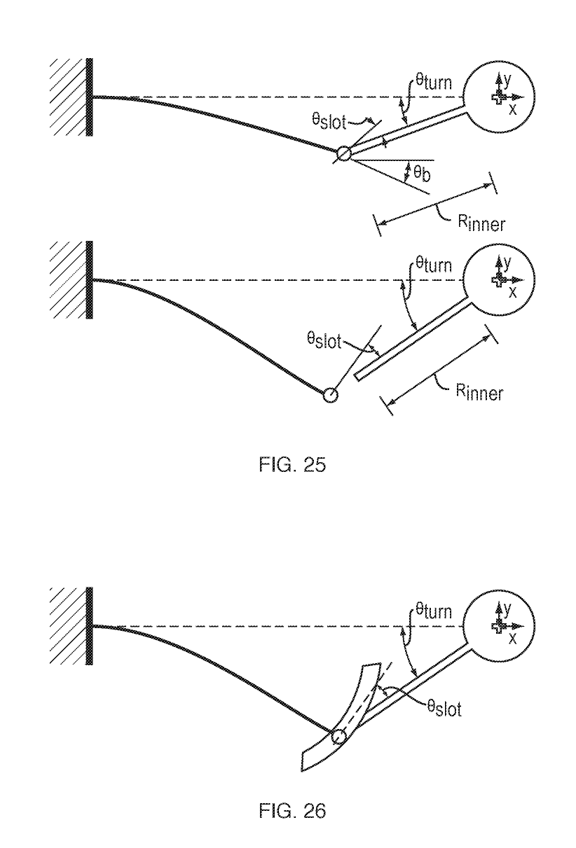

In designing the spring for exoskeleton applications, efficiency is an important factor that should be optimized, especially at higher deflections. In the straight-slot design, higher deflections resulted in drastically lower efficiencies. In attempting to optimize the slot design, the use of a curved slot was explored. As shown in FIG. 25, the curved slot is configured such that at any given angular deflection of the inner hub, the slot at that point is angled θₛₗₒₜ with respect to the radius vector to the beam's end. The curvature of the slot is such that at any given θₜᵤᵣₙ, the angle between the slot at that point and the radius vector, θₛₗₒₜ, is constant. This results in a curved slot design such as the example shown in FIG. 26.

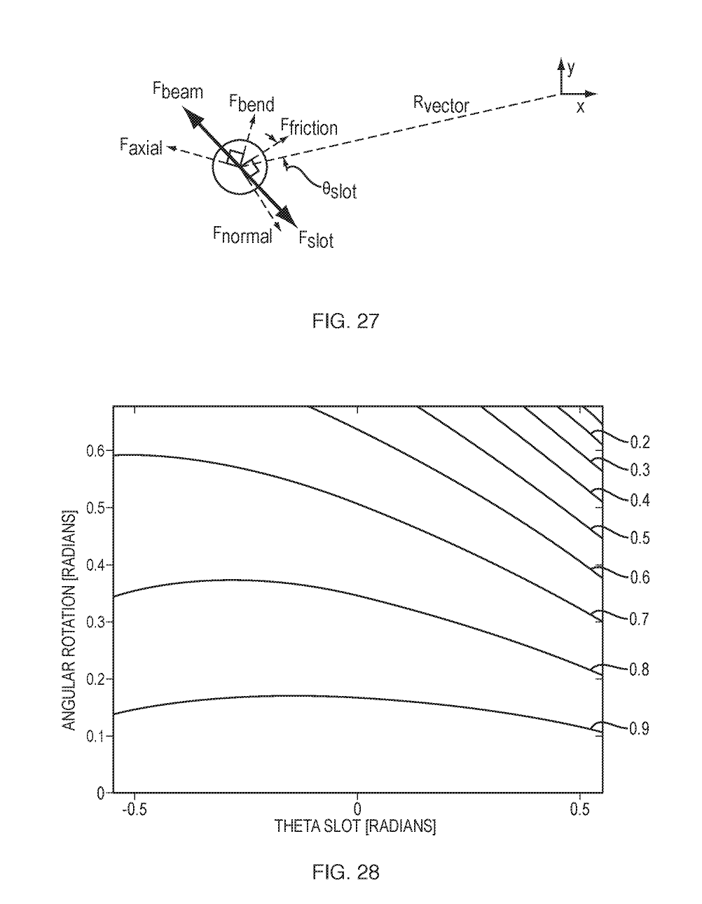

Beam and Slot Forces. In analyzing the forces that act on the pin with a curved slot, the approach was very similar to that of the straight slot modeled in Equations 4.1-4.14, except that, where before the slot was along the same vector as \vec{R}_{vector}, the slot vector is now angled with respect to the radius vector, \vec{R}_{vector}, since the forces are now acting on a slot that is angled with respect to the radius vector. In this curved slot case, the forces \vec{F}_{friction} and \vec{F}ₙₒᵣₘₐₗ now act on the angled slot vector, as shown in FIG. 27.

The slot vector, \vec{C}ₛₗₒₜ, at any specific θₜᵤᵣₙ is the intersection of the beam end trajectory and the line that is rotated about the end of the inner radius by θₛₗₒₜ.

Ĉₓ=−cos(θₜᵤᵣₙ+θₛₗₒₜ) (4.16)

Ĉ_{y}=−sin(θₜᵤᵣₙ+θₛₗₒₜ) (4.17)

Similar to the calculations done for the straight slot, the magnitude and direction is known for \vec{F}_{bend} as 6 increases, but for the \vec{F}ₐₓᵢₐₗ, \vec{F}_{friction}, and \vec{F}ₙₒᵣₘₐₗ vectors, only direction is known. In order to characterize the torque response of the beam, \vec{F}ₛₗₒₜ as a function of angular deflection of the spring must be calculated.

Similar to Equation 4.7, force balance on the slot gives the following:

$$\vert {\vec{F}}_{axial} \vert [ \begin{matrix}- {\hat{F}}_{bend - y} \\ {\hat{F}}_{bend - x}\end{matrix} ] + \vert {\vec{F}}_{slot} \vert [ \begin{matrix}{\hat{F}}_{slot - x} \\ {\hat{F}}_{slot - y}\end{matrix} ] + \vert {\vec{F}}_{bend} \vert [ \begin{matrix}{\hat{F}}_{bend - x} \\ {\hat{F}}_{bend - y}\end{matrix} ] = 0 \qquad ( 4.18 )$$

However, in the curved slot case, the components of \vec{F}ₛₗₒₜ are defined as

\vec{F}ₛₗₒₜ₋ₓ=|\vec{F}ₙₒᵣₘₐₗ|Ĉ_{y}+μ|\vec{F}ₙₒᵣₘₐₗ|Ĉₓ (4.19)

and

\vec{F}_{slot-y}=|\vec{F}ₙₒᵣₘₐₗ|Ĉₓ+μ|\vec{F}ₙₒᵣₘₐₗ|Ĉ_{y} (4.20)

Substituting this into and rearranging Equation 4.18:

$$\begin{matrix}[ \begin{matrix}- {\hat{F}}_{bend - y} & \vert {\vec{F}}_{normal} \vert {\hat{C}}_{y} + μ \vert {\vec{F}}_{normal} \vert {\hat{C}}_{x} \\ {\hat{F}}_{bend - x} & - \vert {\vec{F}}_{normal} \vert {\hat{C}}_{x} + μ \vert {\vec{F}}_{normal} \vert {\hat{C}}_{y}\end{matrix} ] [ \begin{matrix}\vert {\vec{F}}_{axial} \vert \\ \vert {\vec{F}}_{slot} \vert\end{matrix} ] = - \vert {\vec{F}}_{bend} \vert [ \begin{matrix}\vert {\vec{F}}_{bend - x} \vert \\ \vert {\vec{F}}_{bend - y} \vert\end{matrix} ] & ( 4.21 ) \\ and & \\ [ \begin{matrix}\vert {\vec{F}}_{axial} \vert \\ \vert {\vec{F}}_{slot} \vert\end{matrix} ] = {[ \begin{matrix}- {\hat{F}}_{bend - y} & \vert {\vec{F}}_{normal} \vert {\hat{C}}_{y} + μ \vert {\vec{F}}_{normal} \vert {\hat{C}}_{x} \\ {\hat{F}}_{bend - x} & - \vert {\vec{F}}_{normal} \vert {\hat{C}}_{x} + μ \vert {\vec{F}}_{normal} \vert {\hat{C}}_{y}\end{matrix} ]}^{- 1} ( - \vert {\vec{F}}_{bend} \vert ) [ \begin{matrix}\vert {\vec{F}}_{bend - x} \vert \\ \vert {\vec{F}}_{bend - y} \vert\end{matrix} ] & ( 4.22 )\end{matrix}$$

From Equation 4.22, the magnitudes of \vec{F}ₐₓᵢₐₗ and \vec{F}ₛₗₒₜ are calculated, where μ is the coefficient of friction between the pin and the slot. Using this, the entirety of \vec{F}ₛₗₒₜ vector can be calculated for all deflections.

\vec{F}ₛₗₒₜ=−\vec{F}_{bend}−\vec{F}ₐₓᵢₐₗ (4.23)

Efficiency. FIG. 28 is the efficiency contour plot showing the effect of θₜᵤᵣₙ and θₛₗₒₜ on the efficiency of the spring. A θₛₗₒₜ=0 shows the efficiency of the straight slot spring design. In order to calculate the efficiency contour shown in FIG. 28, the efficiency is calculated for turning the spring in one direction and then back to zero for μ=0.2, which is the coefficient of friction for lubricated steel-on-steel contact [Erik Oberg, Franklin D. Jones, Holbrook L. Horton, and Henry H. Ryffel. Machinery's Handbook 29th Edition. Industrial Press, 2012]. Efficiency was calculated by taking the ratio of torque resulting from −μ to torque resulting from μ at each θₜᵤᵣₙ for −0.5 radians<θₛₗₒₜ<0.5 radians. The results shown in FIG. 28 can be used to derive a slot geometry function that optimizes efficiency for a particular range of motion depending on the application.

While the mathematical models for the torsion spring design provide a good foundation, the next step is to create a physical prototype of the straight-slotted spring design and perform testing. Through testing, the actual torque responses and efficiencies can be explored, especially at higher angular rotations, and the model revised as necessary. In addition to improving the model, alternative design features can be explored to further minimize the mass and size of the spring.

While preferred embodiments of the invention are disclosed herein, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention.

What is claimed is:

1. A planar torsion spring, comprising:
an outer hub;
an inner hub; and
a plurality of beams connecting the outer hub to the inner hub, the beams being capable of undergoing sufficient bending to provide torsional compliance when the outer hub is rotated with respect to the inner hub, wherein each beam is fixed to the outer hub at one end of the beam and is attached to the inner hub at the other end of the beam by a respective pin and a slot.

2. The planar torsion spring of claim 1, wherein the slots are curved.

3. The planar torsion spring of claim 1, wherein there are more than two beams.

4. The planar torsion spring of claim 1, wherein the spring is capable of deflecting greater than ±
$$\frac{π}{36}$$
radians.

5. The planar torsion spring of claim 4, wherein the spring is capable of deflecting to at least ±
$$\frac{π}{6}$$
radians.

6. The planar torsion spring of claim 1, wherein the spring is capable of providing at least 100 N·m of torque.

7. The planar torsion spring of claim 1, wherein the spring is made of maraging steel.

8. The planar torsion spring of claim 1, further comprising a bearing located at the interface between each pin and slot.

9. The planar torsion spring of claim 1, wherein at least some of the beams have a variable width along their length.

10. The planar torsion spring of claim 1, wherein at least some of the beams have a different width than other beams.

11. A method for fabricating an application-specific planar torsion spring according to a set of application-based constraints, the torsion spring comprising an inner hub, an outer hub, and a plurality of beams attached between the inner and outer hubs, wherein each beam is fixed to the outer hub at one end of the beam and is attached to the inner hub at the other end of the beam by a respective pin and slot, the method comprising the steps of:
based on the application-based constraints, parameterizing at least some of beam width, beam length, beam thickness, beam material, and slot geometry of the planar torsion spring to obtain a parameterized model that characterizes the effects of the parameters on efficiency, torque response, and deflection;
based on the parameterized model, establishing an initial design;
optimizing the initial design for at least some of weight, size, maximum stresses, stiffness, efficiency, and performance in order to obtain an optimized torsion spring design; and
fabricating the planar torsion spring according to the optimized torsion spring design.

12. The method of claim 11, further comprising the step of adjusting the spring thickness to obtain the desired stiffness and torque.

13. The method of claim 11, wherein the step of optimizing further comprises the step of minimizing the amount of material in the spring while maximizing energy storage.

14. The method of claim 11, wherein the step of optimizing further comprises the step of minimizing the amount of stiffness in loading the spring while maximizing deflection.

15. The method of claim 11, wherein the step of parameterizing further comprises mathematical modeling of beam bending to determine beam boundary conditions that maximize deflection before yielding.

16. The method of claim 15, wherein the beam boundary conditions comprise a fixed, fixed-roller beam, a fixed, pin-roller beam, and a fixed, free beam.

17. The method of claim 11, wherein the step of optimizing further comprises the step of performing analysis on the amount of stress, bending energy, and tensile energy in each beam.

18. The method of claim 11, wherein the step of optimizing further comprises the step of calculating the amount of maximum beam stress when the beams are undergoing both bending and loading by superposition of the axial and bending stresses in each beam.

19. The method of claim 11, wherein the step of optimizing further comprises the step of calculating the stiffness of each beam by taking the numerical derivative of the energy stored in each beam.

20. The method of claim 11, wherein the step of optimizing further comprises the step of calculating the forces acting on at least one of the pins and the slots in order to determine the torque response of the spring.
